(19) United States
(12) Patent Application Publication
(10) Pub. No.: US 2025/0041001 A1
(43) Pub. Date: Feb. 6, 2025

(54) ARTICULATION APPARATUS FOR ROBOTIC SURGICAL TOOL

(71) Applicant: ROEN Surgical, Inc., Daejeon (KR)

(72) Inventors: Un-Je YANG, Daejeon (KR); Duk-Yoo KONG, Daejeon (KR); Duk-Sang KIM, Daejeon (KR); Dae-Sung LEE, Gyeonggi-do (KR); Dong-Ho LEE, Daejeon (KR)

(73) Assignee: ROEN Surgical, Inc., Daejeon (KR)

(21) Appl. No.: 18/706,723

(22) PCT Filed: Nov. 8, 2022
(86) PCT No.: PCT/KR2022/017390

§ 371 Date: May 1, 2024

(30) Foreign Application Priority Data
Nov. 9, 2021 (KR) 10-2021-0153478

(51) Int. Cl. A61B 34/30; A61B 17/29

(52) U.S. Cl. CPC A61B 34/30; A61B 17/29; A61B 2017/2908

(57) ABSTRACT

Proposed is an articulation apparatus for a robotic surgical tool, which is capable of inner rotation and outer bending independent of each other. To this end, the articulation apparatus includes an inner rotation part which is rotated by first driving power, and an outer bending part which is bent by second driving power different from the first driving power.

### TECHNICAL FIELD

The present disclosure relates to an articulation apparatus for a robotic surgical tool, which is capable of inner rotation and outer bending independent of each other.

### BACKGROUND ART

Conventional articulation devices for surgical instruments implement rotation and bending of a surgical instrument through wire driving. In this case, when both the rotation and bending of the surgical instrument are implemented through wire driving, escaping the influence of hysteresis due to wire driving is not possible, which is problematic.

### DOCUMENT OF RELATED ART

(Patent Document 0001) WO 2017-006377

### DISCLOSURE

### Technical Problem

The present disclosure is intended to solve the above problems occurring in the related art. An objective of the present disclosure is to provide an articulation apparatus for a robotic surgical tool that allows rotation on the inside of an articulation joint for a surgical tool and independent bending on the outside.

However, objectives of the present disclosure are not limited to the objective mentioned above, and other objectives not mentioned will be clearly understood by those skilled in the art from the description below.

### Technical Solution

In order to achieve the above mentioned objective, there is provided an articulation apparatus for a robotic surgical tool, the apparatus including: an inner rotation part configured to be rotated by first driving power; an outer bending part configured to be bent by second driving power different from the first driving power.

In addition, the inner rotation part may be disposed inside the outer bending part and may rotate according to the first driving power.

In addition, the first driving power may be generated by a rotation driving portion directly or indirectly connected to a first side of the inner rotation part, whereas the second driving power may be generated by traction of a wire disposed in the outer bending part.

In addition, an end effector portion may be connected directly or indirectly by link connection to a first end of the inner rotation part.

In addition, the inner rotation part may include: an upper axis joint portion in which a first bending axis and a first coupling axis are formed; a lower axis joint portion in which a second bending axis and a second coupling axis are formed; a link portion disposed between the upper axis joint portion and the lower axis joint portion, and fixedly coupled to the upper axis joint portion and the lower axis joint portion on the first and second coupling axes; an upper joint link portion jointly coupled to the upper axis joint portion on the first bending axis; and a lower joint link portion jointly coupled to the lower axis joint portion on the second bending axis.

In addition, the link portion may be combined with the upper and lower axis joint portions in a first direction, and the upper and lower joint link portions may be combined with the upper and lower axis joint portions in a second direction perpendicular to the first direction.

In addition, the upper axis joint portion and the lower axis joint portion may be arranged as a pair to face each other, and the bending axes and the coupling axes may be orthogonal to each other.

In addition, the upper axis joint portion and the lower axis joint portion may respectively include: first coupling axis portions provided on the first and second coupling axes and fixedly coupled to a first side of the link portion by protruding from axis coupling portions in a first direction; and second coupling axis portions provided on the first and second coupling axes and fixedly coupled to a second side of the link portion by protruding from the axis coupling portions in a second direction.

In addition, the upper axis joint portion may include: a first bending axis portion provided on the first bending axis and jointly coupled with a first side of the upper joint link portion by protruding from the axis coupling portion in a third direction; and a second bending axis portion provided on the second bending axis and jointly coupled with a second side of the upper joint link portion by protruding from the axis coupling portion in a fourth direction, and
the lower axis joint portion may include: a first bending axis portion provided on the second bending axis and jointly coupled with a first side of the lower joint link portion by protruding from the axis coupling portion in the third direction; and a second bending axis portion provided on the second bending axis and jointly coupled with a second side of the lower joint link portion by protruding from the axis coupling portion in the fourth direction.

In addition, the link portion may include: first and second upper link portions that protrude from a link coupling portion in a first direction and are respectively fixedly coupled to first and second coupling axis portions of the upper axis joint portion; and first and second lower link portions that protrude from the link coupling portion in a second direction opposite to the first direction and are respectively fixedly coupled to first and second coupling axis portions of the lower axis joint portion.

In addition, the first and second upper link portions and the first and second lower link portions may be provided in symmetrical pairs.

In addition, the upper joint link portion may include upper first and second joint coupling portions formed symmetrically left and right and are respectively jointed with first and second bending axis portions of the upper axis joint portion, and
the lower joint link portion may include lower first and second joint coupling portions formed symmetrically left and right and are respectively jointed with first and second bending axis portions of the lower axis joint portion.

In addition, with respect to the link portion, the upper first and second joint coupling portions and the lower first and second joint coupling portions may be jointed in symmetrical pairs to the upper axis joint portion and the lower axis joint portion, respectively.

In addition, the outer bending part may include: left and right restrain-coupling portions arranged side by side with each other and where a wire that generates the second driving power is fixed; a first bending portion coupled and restrained to first sides of the left and right restrain-coupling portions; and a second bending portion coupled and restrained to second sides of the left and right restrain-coupling portions.

In addition, each of the left and right restrain-coupling portions may be formed to protrude from a restrain-coupling portion, and may include: a first restrain-coupling portion restrain-coupled to the first bending portion; and a second restrain-coupling portion formed to protrude from the restrain-coupling portion at a certain distance from the first restrain-coupling portion, and restrain-coupled to the second bending portion.

In addition, an articulation joint may be bent by a traction force of the wire generated due to a pulley drive of the wire as the wire is sleeve-fixed to the second restrain-coupling portion.

In addition, the inner rotation part is disposed between the left and right restrain-coupling portions.

In addition, each of the first and second bending portions may include: a first gear portion formed symmetrically left and right and is restrain-coupled to the first restrain-coupling portion of the left and right restrain-coupling portions; and a second gear portion formed symmetrically left and right and is restrain-coupled to the second restrain-coupling portion of the left and right restrain-coupling portions.

In addition, depending on a direction of movement of the wire, gear engagement of the first gear portion and the second gear portion may change to bend an articulation joint.

In addition, the inner rotation part may further include: a body disposed inside the outer bending part, rotated according to the first driving power, and made of a flexible material to enable bending.

In addition, the body may have a plurality of slits arranged alternately, wherein the plurality of slits may include: a first slit in which an upper slit formed on an upper part of the body and a lower slit formed on a lower part of the body are arranged to face each other; and a second slit in which a left side slit formed on a left side of the body and a right side slit formed on a right side of the body are arranged to face each other, wherein the first slit and the second slit may be formed alternately along a longitudinal direction of the body.

In addition, the first driving power may be generated by a rotation driving portion directly or indirectly connected to a first side of the inner rotation part, whereas the second driving power may be generated by traction of a wire disposed in the outer bending part, and an end effector portion may be connected directly or indirectly by link connection to a second end of the inner rotation part.

In addition, when the first side of the inner rotation part rotates due to the first driving power, a second side of the inner rotation part may rotate together to generate a rotational force in the end effector portion.

In addition, the end effector portion may be composed of a pair of grasper units, wherein a pair of operating wires respectively connected to the pair of gripper units may be individually driven by a pull operation.

### Advantageous Effects

According to the present disclosure as described above, since the rotational movement of an articulation joint is not implemented by means of a wire, the effect of hysteresis due to wire driving can be reduced.

### DESCRIPTION OF DRAWINGS

The following drawings attached to this specification illustrate preferred embodiments of the present disclosure, and serve to further make the technical idea of the present disclosure understood along with the detailed description of the present disclosure. Thus, the present disclosure should not be construed as limited to the matters described in the drawings.

FIGS. 1 to 3 are views showing an articulation apparatus for a robotic surgical tool according to an embodiment

of the present disclosure;

FIG. 4 is a view showing bending and unfolding of an outer bending part according to an embodiment

of the present disclosure;

FIG. 5 is a view showing rotation of an inner rotation part according to an embodiment of the present disclosure;

FIG. 6 is a view schematically showing the configuration of the inner rotation part according to an embodiment

of the present disclosure;

FIG. 7 is a view showing upper and lower axis joint portions according to an embodiment

of the present disclosure;

FIG. 8 is a view showing a link portion according to an embodiment of the present disclosure;

FIG. 9 is a view showing the fixed coupling of the link portion and the upper and lower axis joint portions according to an embodiment

of the present disclosure;

FIG. 10 is a view showing an upper joint link portion according to an embodiment of the present disclosure;

FIG. 11 is a view showing a lower joint link portion according to an embodiment of the present disclosure;

FIG. 12 is a view schematically showing the configuration of the inner rotation part according to an embodiment

of the present disclosure;

FIG. 13 is a view showing a restrain-coupling portion of the outer bending part according to an embodiment

of the present disclosure;

FIG. 14 is a view showing that first and second wires are sleeve-fixed to the restrain-coupling portion according to

an embodiment of the present disclosure;

FIG. 15 is a view showing the inner rotation part disposed within the restrain-coupling portions according to an embodiment of the present disclosure;

FIG. 16 is a view showing first and second bending portions according to an embodiment of the present disclosure;

FIG. 17 is a view showing the second bending portion being restrain-coupled to a second restrain-coupling portion according to

an embodiment of the present disclosure;

FIG. 18 is a view showing the first bending portion being restrain-coupled to a first restrain-coupling portion according to an embodiment of the present disclosure;

FIG. 19 is a view schematically showing the configuration of an inner rotation part according to another embodiment of the present disclosure;

FIG. 20 is a view showing the bending state of an outer bending part in which an inner rotation part is disposed inside according to another embodiment of the present disclosure;

FIGS. 21 and 22 are views showing rotations of the inner rotation part according to another embodiment of the present disclosure;

FIG. 23 is a view showing a state in which the inner rotation part is installed on the outer bending part according to another embodiment of the present disclosure; and

FIG. 24 is a view showing a state in which an end effector portion operates in a pull-pull manner according to another embodiment of the present disclosure.

### BEST MODE

The present disclosure may be achieved by providing an articulation apparatus for a robotic surgical tool including an inner rotation part that rotates by a first driving power and an outer bending part that is bent by a second driving power different from the first driving power.

### MODE FOR INVENTION

Hereinafter, a preferred embodiment of the present disclosure will be described with reference to the drawings. The embodiment described below does not unduly limit the content of the present disclosure described in the claims, and the entire configuration described in this embodiment is not essential as a solution to the present disclosure. In addition, the description of related art or matters that are obvious to those skilled in the art may be omitted from description, and the descriptions of these omitted components (methods) and functions may be sufficiently referenced without departing from the technical spirit of the present disclosure.

An articulation apparatus for a robotic surgical tool according to an embodiment of the present disclosure relates to an invention in which rotation and bending of a link joint are actuated by different driving powers.

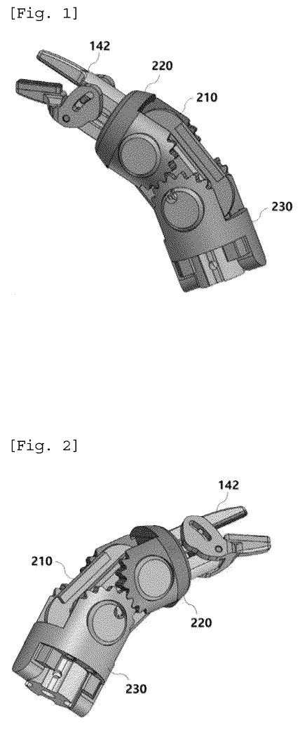

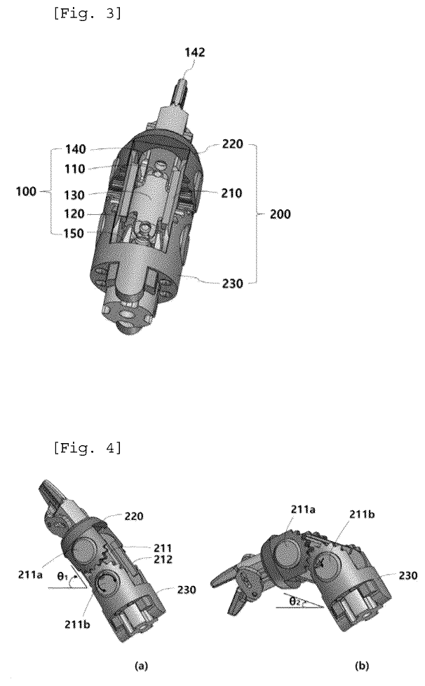

As shown in FIGS. 1 to 3, the articulation apparatus includes an inner rotation part 100 and an outer bending part 200 provided on the outside of the inner rotation part 100. The inner rotation part 100 and the outer bending part 200 rotate and bend respectively by different types of driving powers. To be specific, as shown in FIG. 4, the outer bending part 200 is bent (θ2) or straightened (θ1) depending on the direction of traction or pulling of a driving wire on the outside of the articulation apparatus, and as shown in FIG. 5, the inner rotation part 100 is directly or indirectly connected to the torque tube, which is a rotation driving portion, and rotates inside the articulation apparatus.

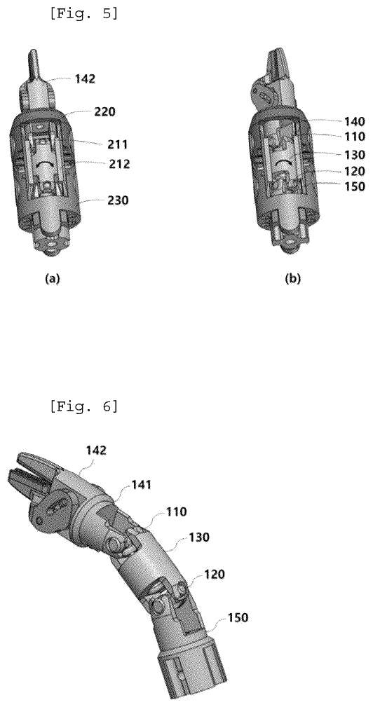

As shown in FIG. 6, the inner rotation part 100 includes upper and lower axis joint portions 110 and 120, a link portion 130, and upper and lower joint link portions 140 and 150.

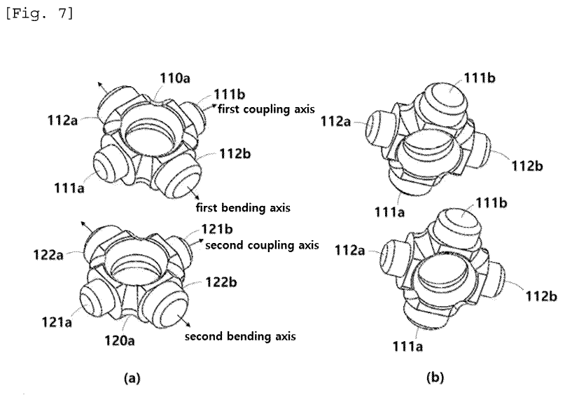

As shown in FIG. 7, the upper axis joint portion 110 and the lower axis joint portion 120 are arranged as a pair in the vertical direction. Thus, the upper axis joint portion 110 and the lower axis joint portion 120 have the same configuration. Hereinafter, only the upper axis joint portion 110 will be described, and the same description of the lower axis joint portion 120 will be replaced with the description of the upper axis joint portion 110.

The upper axis joint portion 110 includes first and second coupling axis portions 11a and 111b and first and second bending axis portions 112a and 112b respectively protruding from an upper axis body portion 110a in the first, second, third and fourth directions. The first and second coupling axis portions 111a and 111b form a first coupling axis while the first and second bending axis portions 112a and 112b form a first bending axis. By the same principle, in the lower axis joint portion 110, a second coupling axis and a second bending axis are formed by first and second coupling axis portions 121a and 121b and first and second bending axis portions 122a and 122b. The first coupling axis and the first bending axis or the second coupling axis and the second bending axis are preferably formed perpendicular to each other.

As shown in FIG. 9, a first upper link portion 131a is fixed coupled to the first coupling axis portion 111a of the upper axis joint portion 110, and a second upper link portion 131b is fixed coupled to the second coupling axis portion 111b of the upper axis joint portion 110. By the same principle, a first lower link portion 132a is fixed coupled to the first coupling axis portion 121a of the lower axis joint portion 120, and a second lower link portion 132b is fixed coupled to the second coupling axis portion 121b of the lower axis joint portion 120.

The upper axis body portion 110a and a lower axis body portion 120a have a cross-section of approximately a “circle or ellipse” shape, and a through hole is formed in the central area of the upper axis body portion 110a and in the central area of the lower axis body portion 120a.

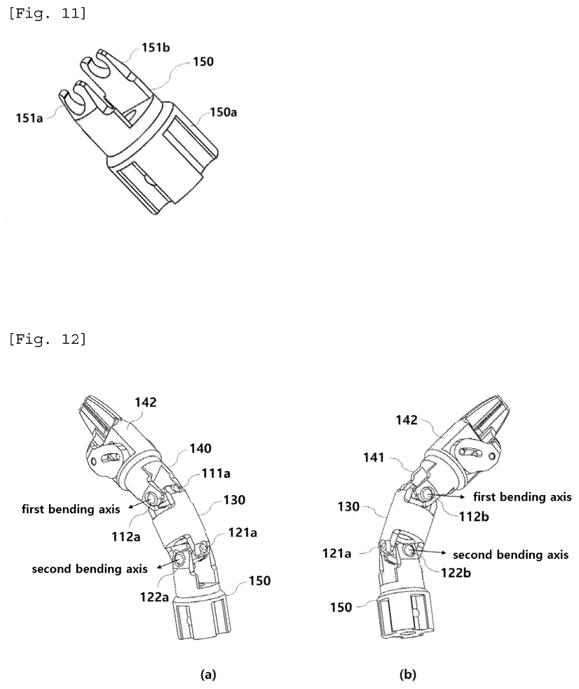

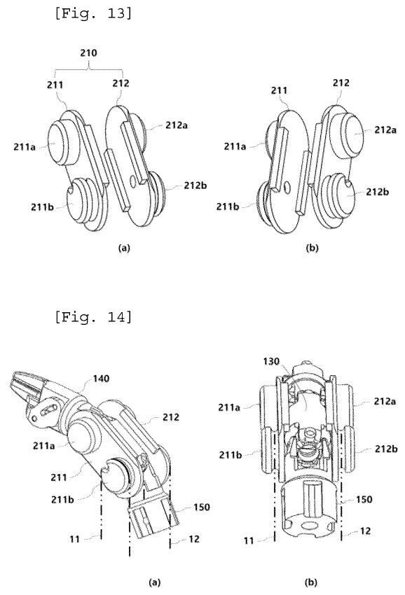

As shown in FIG. 12, an upper first joint coupling portion 141a is joint-coupled with the first bending axis portion 112a of the upper axis joint portion 110 in the upward direction (based on FIG. 12), and an upper second joint coupling portion 141b is joint-coupled with the second bending axis portion 112b of the upper axis joint portion 110 in the upward direction (based on FIG. 12). In addition, by the same principle, a lower first joint coupling portion 151a is joint-coupled with the first bending axis portion 122a of the lower axis joint portion 120 in the downward direction (based on FIG. 12), and a lower second joint coupling portion 151b is joint-coupled with the second bending axis portion 122b of the lower axis joint portion 120 in the downward direction (based on FIG. 12).

As shown in FIG. 12, in the upward direction based on the upper axis joint portion 110, the upper joint link portion 140 is joint-coupled so as to be first-bent when the outer bending part 200 is bent, whereas in the downward direction based on the upper axis joint portion 110, the link portion 130 is fixed coupled.

In addition, in the upward direction based on the lower axis joint portion 120, the link portion 130 is fixed coupled, whereas in the downward direction based on the lower axis joint portion 120, the lower joint link portion 150 is joint-coupled so as to be second-bent when the outer bending part 200 is bent.

Thus, by using two pin joints through the upper axis joint portion 110 and the lower axis joint portion 120 to achieve the first bend and the second bend, when bending the outer bending part 200, a bending angle may be reduced to prevent a sudden radius of curvature from occurring. Due to the two pin joints, a rotation speed of an input end and an output end may also be kept constant when the inner rotation part 100 rotates.

As shown in FIG. 8, the link portion 130 is composed of a top/bottom or left/right symmetrical pair. That is, the link portion 130 is composed of first and second upper link portions 131a and 131b and first and second lower link portions 132a and 132b in a symmetrical pair with respect to a link body portion 130a.

The link body portion 130a has a cross-sectional shape of approximately “circle or ellipse” in the central area thereof, and a through hole is formed in the center.

Referring to FIG. 8, the first and second upper link portions 131a and 131b are formed to protrude from the upper surface of the link body portion 130a in the upward direction at a certain distance from each other, and the first and second lower link portions 132a and 132b are formed to protrude from the lower surface of the link body portion 130a in the downward direction at a certain distance from each other.

At ends of the first and second upper link portions 131a and 131b and the first and second lower link portions 132a and 132b, fixed coupling holes are formed that are respectively coupled to the coupling axis portions 111a, 111b, 121a and 121b.

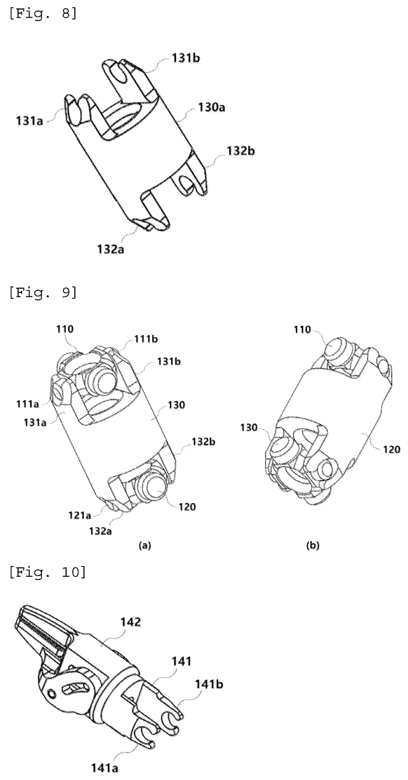

As shown in FIG. 10, the upper joint link portion 140 includes a joint link portion 141 and an end effector portion 142.

The joint link portion 141 includes: an upper first joint coupling portion 141a jointly coupled with the first bending axis portion 112a of the upper axis joint portion 110; and an upper second joint coupling portion 141b jointly coupled with the second bending axis portion 112b of the upper axis joint portion 110.

The upper first and second joint coupling portions 141a and 141b are provided symmetrically left and right at a certain distance from each other to enable joint coupling with the first and second bending axis portions 112a and 112b.

The end effector portion 142 is directly or indirectly connected to one side of the joint link portion 141. Although FIG. 10 shows that the end effector portion 142 is directly connected to the joint link portion 141, an additional connection link may be added in the middle so as to indirectly connect the end effector portion 142 and the joint link portion 141.

As shown in FIG. 11, the lower joint link portion 150 includes: a torque tube coupling portion 150a that is directly or indirectly connected to a torque tube (not shown) that transmits rotational force; and lower first and second joint coupling portions 151a and 151b.

The lower first and second joint coupling portions 151a and 151b protrude upward from the upper surface of the torque tube coupling portion 150a and are symmetrically arranged left and right at a certain distance from each other to enable joint coupling with the first and second bending axis portions 122a and 122b.

Joint coupling holes are formed at the ends of the upper first and second joint coupling portions 141a and 141b and the lower first and second joint coupling portions 151a and 151b to enable joint coupling.

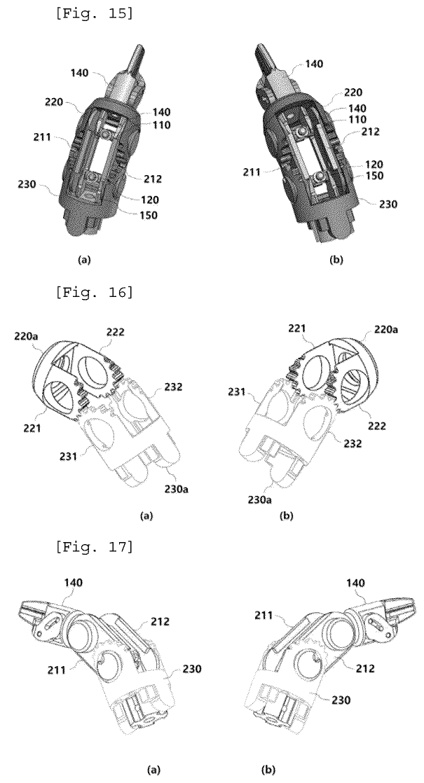

As shown in FIGS. 13 to 18, the outer bending part 200 includes a restrain-coupling portion 210 and first and second bending portions 220 and 230.

As shown in FIG. 13, the restrain-coupling portion 210 is provided in a pair, with one of the pair and the other of the pair being arranged side by side with each other. As shown in FIG. 15, the upper and lower axis joint portions 110 and 120, the link portion 130, and the upper and lower joint link portions 140 and 150 are disposed inside the pair of restrain-coupling portions 210.

The restrain-coupling portion 210 is arranged in a pair with a left restrain-coupling portion 211 and a right restrain-coupling portion 212 side by side. Thus, hereinafter, only the left restrain-coupling portion 211 will be described, and the same description of the right restrain-coupling portion 212 will be replaced with the description of the left restrain-coupling portion 211.

The left restrain-coupling portion 211 includes first and second restrain-coupling portions 211a and 211b. The first restrain-coupling portion 211a protrudes from the bottom surface of the body of the left restrain-coupling portion 211 and has a cross-sectional shape of a “circle or ellipse”. The second restrain-coupling portion 211b protrudes from the bottom surface of the body of the left restrain-coupling portion 211 at a certain distance from the first restrain-coupling portion 211a, and has a cross-sectional shape of a “circle or ellipse”.

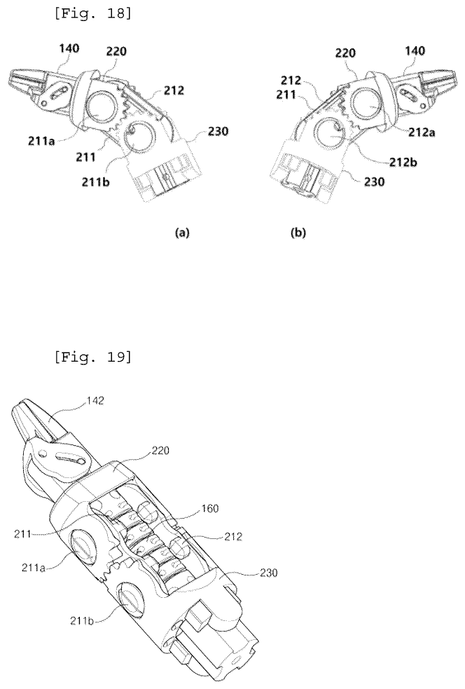

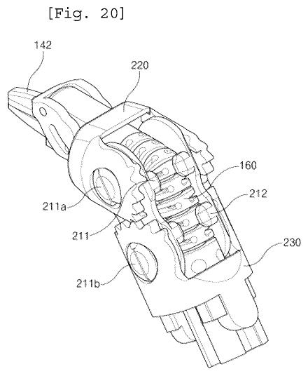

As shown in FIG. 18, the first restrain-coupling portions 211a and 212a are restrain-coupled to the first bending portion 220, and the second restrain-coupling portions 211b and 212b are restrain-coupled to the second bending portion 230.

As shown in FIG. 14, a driving wire is sleeve-fixed to the second restrain-coupling portions 211b and 212b so that the driving wire may be pulley driven. A first pulley driving portion (not shown) pulley drives a first wire 11 in the forward/reverse direction, and a second pulley driving portion (not shown) pulley drives a second wire 12 in the forward/reverse direction, thereby adjusting an bending angle according to the rotation direction of a pulley. At this time, it is preferable that the first and second pulley driving portions are synchronized with each other and drive the wires in the same direction.

As shown in FIG. 16, the first bending portion 220 includes first and second gear portions 221 and 222. The first gear portion 221 includes a first restrain-coupling hole to be restrained and coupled to the first restrain-coupling portion 211a of the left restrain-coupling portion 211, and the second gear portion 222 includes a second restrain-coupling hole to be restrained and coupled to the first restrain-coupling portion 212a of the right restrain-coupling portion 212. The first gear portion 221 and the second gear portion 222 are arranged left/right symmetrical to each other and protrude in one direction from a body portion 220a.

The second bending portion 230 includes first and second gear portions 231 and 232. The first gear portion 231 includes a first restrain-coupling hole to be restrained and coupled to the second restrain-coupling portion 211b of the left restrain-coupling portion 211, and the second gear portion 232 includes a second restrain-coupling hole to be restrained and coupled to the second restrain-coupling portion 212b of the right restrain-coupling portion 212. The first gear portion 231 and the second gear portion 232 are arranged left/right symmetrical to each other and protrude in one direction from a body portion 230a.

Gears formed on the end surfaces of the first and second gear portions 221 and 222 of the first bending portion 220 and the first and second gear portions 231 and 232 of the second bending portion 230 are respectively engaged with each other, and the bending angle changes as the gear mesh changes depending on the traction force of a driving wire.

As shown in FIG. 19, the inner rotation part 100 further includes a body 160 disposed inside the outer bending part 200, rotated according to the first driving power, and having a plurality of slits 161 interlaced with each other.

Since the body 160 160 has a shape in which the slits 161 arranged alternately and is made of a flexible material, the body 160 may also be bent together when the first bending portion 220 and the second bending portion 230 are bent by gear engagement.

In addition, the slits 161 include: a first slit 162 in which an upper slit 162a formed on the upper part of the body 160 and a lower slit 162b formed on the lower part of the body 160 are arranged to face each other; and a second slit 163 in which a left side slit 163a formed on the left side of the body 160 and a right side slit 163b formed on the right side of the body 160 are arranged to face each other. The first slits 162 and the second slits 163 are formed alternately along the longitudinal direction of the body 160, so that the body 160 may be flexibly bent during the bending operation of the outer bending part 200.

The first driving power is generated by the rotation driving portion directly or indirectly connected to one side of the inner rotation part 100, and the end effector portion 142 is connected directly or indirectly by link connection to the other end of the inner rotation part 100.

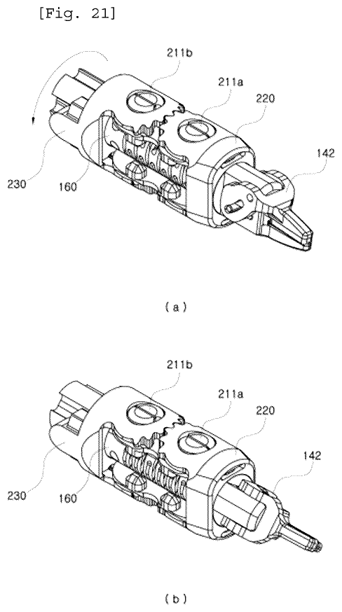

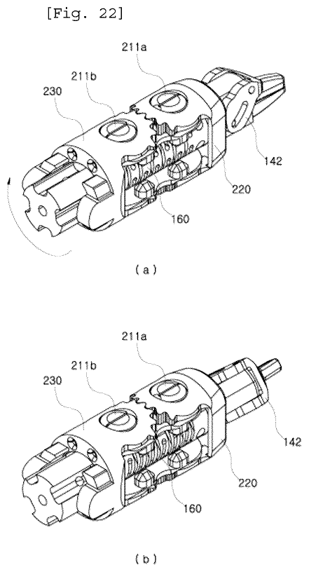

As shown in FIGS. 21 and 22, when one side of the inner rotation part 100 connected to the rotation driving portion rotates by the first driving power, the other side of the inner rotation part 100 rotates together and generates a rotational force in the end effector portion 142, and accordingly, the linked end effector portion 142 rotates together, thereby enabling rotational actuation of a link joint.

In addition, even in a bent state due to gear engagement of the outer bending part 200, when the inner rotation part 100 rotates due to the first driving power, the rotational force is transmitted to the end effector portion 142, enabling rotational actuation.

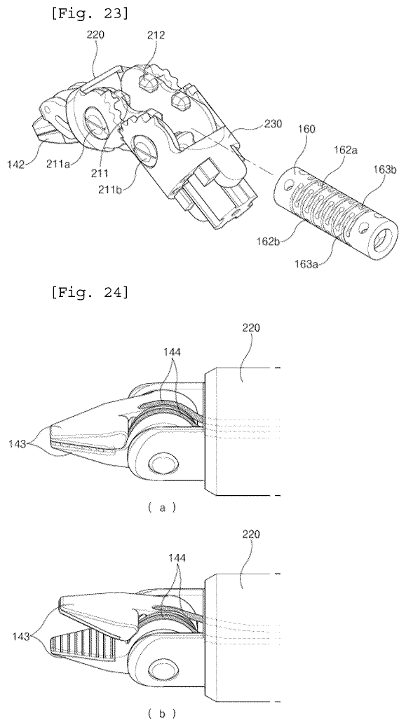

FIG. 23 is a view showing a state in which the inner rotation part 100 is installed on the outer bending part 200. Although it is difficult for the inner rotation part 100 to be installed inside the outer bending part 200 due to the left restrain-coupling portion 211 and the right restrain-coupling portion 212 disposed on opposite sides of the restrain-coupling portion 210, the inner rotation part 100 may be installed inside the outer bending part 200 by inserting the inner rotation part 100 while the outer bending part 200 is bent at a certain bending angle by gear engagement of the first bending portion 220 and the second bending portion 230.

As shown in FIG. 24, the end effector portion 142 is composed of a pair of grasper units 143, and the pair of grasper units 143 are rotatable about a rotation axis and are adjacent to each other to perform a grasp operation.

In this case, a single operating wire 144 is connected to the pair of grasper units 143 so as to be wound around the rotation axis. The grasp operation may be performed using a push-pull method in which when one side of the operating wire 144 is pulled in the opposite direction to the end effector portion 142, the other side of the operating wire 144 is pushed in the direction of the end effector portion 142.

In addition, as shown in FIG. 24, the operating wire 144 may be connected to each of the grasper units 143, and the grasp operation may be performed by pulling each of the operating wires 144.

The pair of grasper units 143 is an embodiment of the end effector portion 142. The configuration of the end effector portion is not limited thereto, and surgical tools such as scissors, dissectors, forceps, bipolars, clips, needles, sealers, etc. that are expected to increase surgical convenience through inner rotation may also form the end effector portion 142.

In describing the present disclosure, the description of related art or matters that are obvious to those skilled in the art may be omitted from description, and the descriptions of these omitted components (methods) and functions may be sufficiently referenced without departing from the technical spirit of the present disclosure. In addition, the components of the present disclosure described above have been described for the convenience of explaining the present disclosure, and components not described herein may be added without departing from the technical spirit of the present disclosure.

The description of the configuration and function of each part (portion) described above is provided separately for convenience of explanation, and as needed, one configuration and function may be implemented by integrating with other components, or may be implemented in further detail.

Although the present disclosure has been described above with reference to an embodiment, the present disclosure is not limited thereto, and various modifications and applications are possible. That is, those skilled in the art will easily understand that many modifications are possible without departing from the gist of the present disclosure. Furthermore, it should be noted that if it is determined that a detailed description of the known functions and their configurations related to the present disclosure or the combination relationship between each component of the present disclosure may unnecessarily obscure the gist of the present disclosure, the detailed description has been omitted.

### DESCRIPTION OF NUMERALS

11: first wire
12: second wire
100: inner rotation part or inner link part
110: upper axis joint portion
110a: upper axis body portion
111a: first coupling axis portion
111b: second coupling axis portion
112a: first bending axis portion
112b: second bending axis portion
120: lower axis joint portion
120a: lower axis body portion
121a: first coupling axis portion
121b: second coupling axis portion
122a: first bending axis portion
122b: second bending axis portion
130: link portion
130a: link body portion
131a: first upper link portion
131b: second upper link portion
132a: first lower link portion
132b: second lower link portion
140: upper joint link portion
141: joint link portion
141a: upper first joint coupling portion
141b: upper second joint coupling portion
142: end effector portion
143: grasper unit
144: operating wire
150: lower joint link portion
150a: torque tube coupling portion
151a: lower first joint coupling portion
151b: lower second joint coupling portion
160: body
161: slit
162: first slit
162a: upper slit
162b: lower slit
163: second slit
163a: left side slit
163b: right side slit
200: outer bending part
210: restrain-coupling portion
211: left restrain-coupling portion
211a: first restrain-coupling portion
211b: second restrain-coupling portion
212: right restrain-coupling portion
212a: first restrain-coupling portion
212b: second restrain-coupling portion
220: first bending portion
220a: body portion
221: first gear portion
222: second gear portion
230: second bending portion
230a: body portion
231: first gear portion
232: second gear portion

1. An articulation apparatus for a robotic surgical tool, the apparatus comprising:
an inner rotation part configured to be rotated by first driving power; and
an outer bending part configured to be bent by second driving power different from the first driving power.

2. The apparatus of claim 1, wherein the inner rotation part is disposed inside the outer bending part and rotates according to the first driving power.

3. The apparatus of claim 2, wherein the first driving power is generated by a rotation driving portion directly or indirectly connected to a first side of the inner rotation part, whereas the second driving power is generated by traction of a wire disposed in the outer bending part.

4. The apparatus of claim 2, wherein an end effector portion is connected directly or indirectly by link connection to a first end of the inner rotation part.

5. The apparatus of claim 1, wherein the inner rotation part comprises:
an upper axis joint portion in which a first bending axis and a first coupling axis are formed;
a lower axis joint portion in which a second bending axis and a second coupling axis are formed;
a link portion disposed between the upper axis joint portion and the lower axis joint portion, and fixedly coupled to the upper axis joint portion and the lower axis joint portion on the first and second coupling axes;
an upper joint link portion jointly coupled to the upper axis joint portion on the first bending axis; and
a lower joint link portion jointly coupled to the lower axis joint portion on the second bending axis.

6. The apparatus of claim 5, wherein the link portion is combined with the upper and lower axis joint portions in a first direction, and the upper and lower joint link portions are combined with the upper and lower axis joint portions in a second direction perpendicular to the first direction.

7. The apparatus of claim 5, wherein the upper axis joint portion and the lower axis joint portion are arranged as a pair to face each other, and the bending axes and the coupling axes are orthogonal to each other.

8. The apparatus of claim 5, wherein the upper axis joint portion and the lower axis joint portion respectively comprise:
first coupling axis portions provided on the first and second coupling axes and fixedly coupled to a first side of the link portion by protruding from axis coupling portions in a first direction; and
second coupling axis portions provided on the first and second coupling axes and fixedly coupled to a second side of the link portion by protruding from the axis coupling portions in a second direction.

9. The apparatus of claim 8, wherein the upper axis joint portion comprises:
a first bending axis portion provided on the first bending axis and jointly coupled with a first side of the upper joint link portion by protruding from the axis coupling portion in a third direction; and
a second bending axis portion provided on the second bending axis and jointly coupled with a second side of the upper joint link portion by protruding from the axis coupling portion in a fourth direction, and
the lower axis joint portion comprises:
a first bending axis portion provided on the second bending axis and jointly coupled with a first side of the lower joint link portion by protruding from the axis coupling portion in the third direction; and
a second bending axis portion provided on the second bending axis and jointly coupled with a second side of the lower joint link portion by protruding from the axis coupling portion in the fourth direction.

10. The apparatus of claim 5, wherein the link portion comprises:
first and second upper link portions that protrude from a link coupling portion in a first direction and are respectively fixedly coupled to first and second coupling axis portions of the upper axis joint portion; and
first and second lower link portions that protrude from the link coupling portion in a second direction opposite to the first direction and are respectively fixedly coupled to first and second coupling axis portions of the lower axis joint portion.

11. The apparatus of claim 10, wherein the first and second upper link portions and the first and second lower link portions are provided in symmetrical pairs.

12. The apparatus of claim 5, wherein the upper joint link portion comprises upper first and second joint coupling portions formed symmetrically left and right and are respectively jointed with first and second bending axis portions of the upper axis joint portion, and
the lower joint link portion comprises lower first and second joint coupling portions formed symmetrically left and right and are respectively jointed with first and second bending axis portions of the lower axis joint portion.

13. The apparatus of claim 12, wherein with respect to the link portion, the upper first and second joint coupling portions and the lower first and second joint coupling portions are jointed in symmetrical pairs to the upper axis joint portion and the lower axis joint portion, respectively.

14. The apparatus of claim 1, wherein the outer bending part comprises:
left and right restrain-coupling portions arranged side by side with each other and where a wire that generates the second driving power is fixed;
a first bending portion coupled and restrained to first sides of the left and right restrain-coupling portions; and
a second bending portion coupled and restrained to second sides of the left and right restrain-coupling portions.

15. The apparatus of claim 14, wherein each of the left and right restrain-coupling portions is formed to protrude from a restrain-coupling portion, and comprises:
a first restrain-coupling portion restrain-coupled to the first bending portion; and
a second restrain-coupling portion formed to protrude from the restrain-coupling portion at a certain distance from the first restrain-coupling portion, and restrain-coupled to the second bending portion.

16. The apparatus of claim 15, wherein an articulation joint is bent by a traction force of the wire generated due to a pulley drive of the wire as the wire is sleeve-fixed to the second restrain-coupling portion.

17. The apparatus of claim 14, wherein the inner rotation part is disposed between the left and right restrain-coupling portions.

18. The apparatus of claim 15, wherein each of the first and second bending portions comprises:
a first gear portion formed symmetrically left and right and is restrain-coupled to the first restrain-coupling portion of the left and right restrain-coupling portions; and
a second gear portion formed symmetrically left and right and is restrain-coupled to the second restrain-coupling portion of the left and right restrain-coupling portions.

19. The apparatus of claim 18, wherein depending on a direction of movement of the wire, gear engagement of the first gear portion and the second gear portion changes to bend an articulation joint.

20. The apparatus of claim 1, wherein the inner rotation part further comprises:
a body disposed inside the outer bending part, rotated according to the first driving power, and made of a flexible material to enable bending.

21. The apparatus of claim 20, wherein the body has a plurality of slits arranged alternately,
wherein the plurality of slits comprises:
a first slit in which an upper slit formed on an upper part of the body and a lower slit formed on a lower part of the body are arranged to face each other; and
a second slit in which a left side slit formed on a left side of the body and a right side slit formed on a right side of the body are arranged to face each other,
wherein the first slit and the second slit are formed alternately along a longitudinal direction of the body.

22. The apparatus of claim 20, wherein the first driving power is generated by a rotation driving portion directly or indirectly connected to a first side of the inner rotation part, whereas the second driving power is generated by traction of a wire disposed in the outer bending part, and an end effector portion is connected directly or indirectly by link connection to a second end of the inner rotation part.

23. The apparatus of claim 22, wherein when the first side of the inner rotation part rotates due to the first driving power, a second side of the inner rotation part rotates together to generate a rotational force in the end effector portion.

24. The apparatus of claim 4, wherein the end effector portion is composed of a pair of grasper units, wherein a pair of operating wires respectively connected to the pair of gripper units are individually driven by a pull operation.
